(19) United States
(12) Patent Application Publication
(10) Pub. No.: US 2015/0119398 A1
(43) Pub. Date: Apr. 30, 2015

(54) FORM 2 POLYMORPH OF 7-(TERT-BUTYL-D9)-3-(2,5-DIFLUOROPHENYL)-6-((1-METHYL-1H-1,2,4-TRIAZOL-5-YL)METHOXY)-[1,2,4]TRIAZOLO[4,3-B]PYRIDAZINE

(71) Applicant: Concert Pharmaceuticals Inc., Lexington, MA (US)

(72) Inventors: Steve Weissman, Lexington, MA (US); Joanna Bis, Lexington, MA (US); David Turnquist, Lexington, MA (US); David Igo, Lexington, MA (US)

(21) Appl. No.: 14/400,177

(22) PCT Filed: May 11, 2013
(86) PCT No.: PCT/US2013/040688

§ 371 Date: Nov. 10, 2014

Related U.S. Application Data
(60) Provisional application No. 61/646,255, filed on May 11, 2012.

(51) Int. Cl. C07D 487/04

(52) U.S. Cl. CPC C07D 487/04
USPC 514/248; 544/236

(57) ABSTRACT

The present invention provides the Form 2 crystalline polymorph of 7-(ieri-Butyl-d9)-3-(2,5-difluorophenyl)-6((1-methyl-1H-1,2,4-triazol-5-yl)methoxy)-[1,2,4]triazolo[4,3-b]pyridazine. The polymorph disclosed herein is characterized according to one or more of (a) powder X-ray diffraction data (“XRPD”); (b) differential scanning calorimetry (“DSC”); (c) FT-Raman spectrum; (d) FT-IT spectrum; and (e) thermogravimetric analysis (TGA).

### RELATED APPLICATIONS

This application claims the benefit of and priority to U.S. Provisional Patent Application No. 61/646,255, filed May 11, 2012, the contents of which are incorporated herein by reference.

### BACKGROUND OF THE INVENTION

The compound 7-(tert-Butyl)-3-(2,5-difluorophenyl)-6-((1-methyl-1H-1,2,4-triazol-5-yl)methoxy)-[1,2,4]triazolo[4,3-b]pyridazine, also known as L-838417, is a GABA-A receptor antagonist of α1 subtypes, and a functionally selective allosteric agonist of the α2, α3 and α5 subtypes. L-838417 is a preclinical candidate for central nervous system disorders. 7-(tert-Butyl-d₉)-3-(2,5-difluorophenyl)-6-((1-methyl-1H-1,2,4-triazol-5-yl)methoxy)-[1,2,4]triazolo[4,3-b]pyridazine is a deuterated form of L-838417. This deuterated form of L-838417 is Compound 103 described in United States patent publication No. 2010/0056529 at paragraphs [0099]-[0102], which is incorporated by reference herein, and has the Formula I:

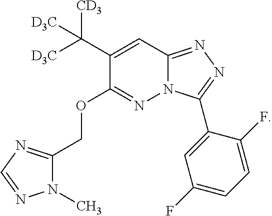 [SMILES: [2H]C([2H])([2H])C(c1cc2nnc(-c3cc(F)ccc3F)n2nc1OCc1ncnn1C)(C([2H])([2H])[2H])C([2H])([2H])[2H]]

It is well known that the crystalline polymorph form of a particular drug is often an important determinant of the drug's ease of preparation, stability, solubility, storage stability, ease of formulation and in vivo pharmacology. Polymorphic forms occur where the same composition of matter crystallizes in a different lattice arrangement resulting in different thermodynamic properties and stabilities specific to the particular polymorph form. In cases where two or more polymorph substances can be produced, it is desirable to have a method to make both polymorphs in pure form. In deciding which polymorph is preferable, the numerous properties of the polymorphs must be compared and the preferred polymorph chosen based on the many physical property variables. It is entirely possible that one polymorph form can be preferable in some circumstances where certain aspects such as ease of preparation, stability, etc. are deemed to be critical. In other situations, a different polymorph maybe preferred for greater solubility and/or superior pharmacokinetics.

Because improved drug formulations, showing, for example, better bioavailability or better stability are consistently sought, there is an ongoing need for new or purer polymorphic forms of existing drug molecules. The crystalline polymorph of 7-(tert-Butyl-d₉)-3-(2,5-difluorophenyl)-6-((1-methyl-1H-1,2,4-triazol-5-yl)methoxy)-[1,2,4]triazolo[4,3-b]pyridazine described herein helps meet these and other needs.

### BRIEF DESCRIPTION OF THE DRAWINGS

FIG. 1 depicts the normalized powder X-ray diffraction pattern of Form 2 of 7-(tert-Butyl-d9)-3-(2,5-difluorophenyl)-6-((1-methyl-1H-1,2,4-triazol-5-yl)methoxy)-[1,2,4]triazolo[4,3-b]pyridazine with the diffraction angles from 0 to 40 degrees.

FIG. 2 depicts the differential scanning calorimetry (“DSC”) thermogram of Form 2 of 7-(tert-Butyl-d9)-3-(2,5-difluorophenyl)-6-((1-methyl-1H-1,2,4-triazol-5-yl)methoxy)-[1,2,4]triazolo[4,3-b]pyridazine (top line); and the corresponding change in weight of the material as temperature increases (bottom line).

FIG. 3 depicts the FT-Raman spectrum of Form 2 of 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

FIG. 4 depicts the FT-IR spectrum of Form 2 of 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

FIG. 5 depicts the DVS isotherm plot of Form 2 of 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

### SUMMARY OF THE INVENTION

The present invention provides a crystalline polymorph of optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine having one or more of the (i) powder X-ray diffraction peaks, and (ii) differential scanning endotherms that are disclosed herein for the crystalline polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine, which is designated as Form 2. The Form 2 polymorph disclosed herein is characterized according to (a) powder X-ray diffraction data (“XRPD”); and (b) differential scanning calorimetry (“DSC”) data. In addition, FT-Raman spectroscopy, FT-infrared spectroscopy, and DVS isotherm plots for the Form 2 polymorph are disclosed.

In one embodiment, the invention is directed to the Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine. In one aspect of this embodiment, the Form 2 polymorph is substantially free of other forms of L-838417, including other crystalline forms such as the other crystalline forms disclosed herein and amorphous forms, of 7-(tert-Butyl-d9)-3-(2,5-difluorophenyl)-6-((1-methyl-1H-1,2,4-triazol-5-yl)methoxy)-[1,2,4]triazolo[4,3-b]pyridazine. Here “other forms” includes other crystalline forms (e.g., Forms 1, 3, 4 and 5 (disclosed herein)), as well as 7-(tert-Butyl-d9)-3-(2,5-difluorophenyl)-6-((1-methyl-1H-1,2,4-triazol-5-yl)methoxy)-[1,2,4]triazolo[4,3-b]pyridazine in amorphous form. In this aspect, the term “substantially free of other forms” means that the sum of the amounts of other forms of is less than 50%, more preferably equal to or less than 20%, more preferably equal to or less than 10%, more preferably equal to or less than 5%, more preferably equal to or less than 1%, or more preferably equal to or less than 0.1%, of the amount of the Form 2 polymorph.

The present invention also provides hydrated solid deuterated 7-(tert-Butyl)-3-(2,5-difluorophenyl)-6-((1-methyl-1H-1,2,4-triazol-5-yl)methoxy)-[1,2,4]triazolo[4,3-b]pyridazine. The invention also provides hydrated crystalline deuterated 7-(tert-Butyl)-3-(2,5-difluorophenyl)-6-((1-methyl-1H-1,2,4-triazol-5-yl)methoxy)-[1,2,4]triazolo[4,3-b]pyridazine.

The present invention further provides a hydrated solid form of 7-(tert-Butyl-d₉)-3-(2,5-difluorophenyl)-6-((1-methyl-1H-1,2,4-triazol-5-yl)methoxy)-[1,2,4]triazolo[4,3-b]pyridazine. In certain embodiments, the form is a crystalline form. In certain embodiments, the crystalline form is the Form 2 polymorph of 7-(tert-Butyl-d₉)-3-(2,5-difluorophenyl)-6-((1-methyl-1H-1,2,4-triazol-5-yl)methoxy)-[1,2,4]triazolo[4,3-b]pyridazine. In certain embodiments, the molar ratio of water to 7-(tert-Butyl-d₉)-3-(2,5-difluorophenyl)-6-((1-methyl-1H-1,2,4-triazol-5-yl)methoxy)-[1,2,4]triazolo[4,3-b]pyridazine (e.g., in certain embodiments, the molar ratio of water to all forms of 7-(tert-Butyl-d₉)-3-(2,5-difluorophenyl)-6-((1-methyl-1H-1,2,4-triazol-5-yl)methoxy)[1,2,4]triazolo[4,3-b]pyridazine; in other embodiments, the molar ratio of water to Form 2 polymorph of 7-(tert-Butyl-d₉)-3-(2,5-difluorophenyl)-6-((1-methyl-1H-1,2,4-triazol-5-yl)methoxy)-[1,2,4]triazolo[4,3-b]pyridazine) is between 0.2 and 2.4. In certain embodiments, the molar ratio of water to 7-(tert-Butyl-d₉)-3-(2,5-difluorophenyl)-6-((1-methyl-1H-1,2,4-triazol-5-yl)methoxy)-[1,2,4]triazolo[4,3-b]pyridazine is between 0.4 and 1.2. In certain embodiments, the molar ratio of water to 7-(tert-Butyl-d₉)-3-(2,5-difluorophenyl)-6-((1-methyl-1H-1,2,4-triazol-5-yl)methoxy)-[1,2,4]triazolo[4,3-b]pyridazine is between 0.6 and 1.0. In certain embodiments, the molar ratio of water to 7-(tert-Butyl-d₉)-3-(2,5-difluorophenyl)-6-((1-methyl-1H-1,2,4-triazol-5-yl)methoxy)-[1,2,4]triazolo[4,3-b]pyridazine is between 0.65 and 0.8. In certain embodiments, the molar ratio of water to 7-(tert-Butyl-d₉)-3-(2,5-difluorophenyl)-6-((1-methyl-1H-1,2,4-triazol-5-yl)methoxy)-[1,2,4]triazolo[4,3-b]pyridazine is about 0.7.

The present invention further provides compositions comprising the Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine. In one embodiment, such compositions are pharmaceutically acceptable compositions additionally comprising a pharmaceutically acceptable carrier.

The present invention further provides a method of treating a mammal having a disorder of the central nervous system, including anxiety and convulsions; and neuropathic, inflammatory and migraine-associated pain, comprising administering to the mammal a therapeutically effective amount of the Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

The present invention further provides methods of synthesizing the Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

The present invention further provides the Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine prepared by any of the methods described herein.

As a hydrate, Form 2 can be distinguished from anhydrous polymorphs of the compound of Formula I based on chemical composition. Analytical data, separate or in combination with chemical composition data corresponding to Form 2, may be used to characterize the Form. Such data may include solid-state techniques such as XRPD and DSC as well as techniques used to detect water content.

In one embodiment, the polymorphs of 7-(tert-Butyl-d9)-3-(2,5-difluorophenyl)-6-((1-methyl-1H-1,2,4-triazol-5-yl)methoxy)-[1,2,4]triazolo[4,3-b]pyridazine disclosed herein are in isolated form.

### DEFINITIONS

The term “Form 2 of 7-(tert-Butyl-d9)-3-(2,5-difluorophenyl)-6-((1-methyl-1H-1,2,4-triazol-5-yl)methoxy)[1,2,4]triazolo[4,3-b]pyridazine” refers to the Form 2 crystalline polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine. The terms “Form 2 of 7-(tert-Butyl-d9)-3-(2,5-difluorophenyl)-6-((1-methyl-1H-1,2,4-triazol-5-yl)methoxy)-[1,2,4]triazolo[4,3-b]pyridazine”, “Form 2”, and “the Form 2 polymorph” are used interchangeably herein.

When the term “7-(tert-Butyl-d9)-3-(2,5-difluorophenyl)-6-((1-methyl-1H-1,2,4-triazol-5-yl)methoxy)[1,2,4]triazolo[4,3-b]pyridazine” or “solid deuterated 7-(tert-Butyl)-3-(2,5-difluorophenyl)-6-((1-methyl-1H-1,2,4-triazol-5-yl)methoxy)-[1,2,4]triazolo[4,3-b]pyridazine” is used without specifying the crystalline form (such as Form 2), this term refers to the compound in any form, such as crystalline, amorphous, or other, or in a combination of forms.

Throughout this application, unless otherwise specified, when a particular position is designated as having deuterium, it is understood that the abundance of deuterium at that position has a minimum isotopic enrichment factor of at least 3340 (50.1% deuterium incorporation) at each atom designated as deuterium in said compound. Preferably, the percentage of deuterium incorporation is at least 80%, at least 85%, at least 90%, at least 95%, at least 98%, or at least 99%.

### EXPERIMENTAL

X-ray powder diffraction (XRPD) data were obtained using a PANalytical X'Pert Pro diffractometer on Si zero-background wafers. All diffractograms were collected using a monochromatic Cu Kα (45 kV/40 mA), with a wavelength of 1.540598 A radiation and a step size of 0.02° 2θ.

Differential Scanning calorimetry (DSC) was conducted with a TA Instruments Q100 differential scanning calorimeter equipped with an autosampler and a refrigerated cooling system under 40 mL/min N₂ purge. DSC thermograms were obtained at 15° C./min in crimped aluminum pans.

FT-IR Spectroscopy. IR spectra were collected with a Nicolet 6700 spectrometer (Thermo Electron) equipped with a DTGS detector and a SensiIR DuroScope DATR. All spectra were acquired at 4 cm⁻¹ resolution, 64 scans, using Happ-Genzel apodization function and 2-level zero-filling.

Dynamic Vapor Sorption (DVS). DVS experiments were conducted on a Surface Measurement Systems DVS-HT at 25° C. The instrument was operated in step mode and the relative humidity was increased in 10% RH increments from 40% RH to 90% RH, then decreased from 90% RH to 0% RH, then increased from 0% RH to 90% RH, then decreased from 90% RH to 0% RH. An extra step at 75% RH was included in each cycle. The mass equilibrium criterion was set at 0.005% change in mass over time (dm/dt) prior to each humidity level. A minimum step time of 10 minutes and a maximum step time of 240 minutes were specified.

FT-Raman Spectroscopy. Raman spectra were collected with a Nicolet NXR9650 or NXR 960 spectrometer (Thermo Electron) equipped with 1064 nm Nd:YVO₄ excitation laser, InGaAs and liquid-N₂ cooled Ge detectors, and a MicroStage. All spectra were acquired at 4 cm⁻¹ resolution, 64-128 scans, using Happ-Genzel apodization function and 2-level zero-filling.

It is common to those of ordinary skill in the art recite x-ray diffraction peaks in approximate terms such as by using the word “about” or “approximately” prior to the peak value in ° 2θ which typically presents the data to within 0.1 or 0.2° 2θ of the stated peak value depending on the circumstances. As used herein, the word “about” or “approximately” when preceding a plurality of values is intended to apply to each number. For the sake of illustration, “about 7.95, 10.09, 10.41, . . . ” means “about 7.95, about 10.09, about 10.41, . . . ”. For purposes herein, “about” is meant to be on the order of plus or minus 0.2° 2θ under typical conditions.

As used herein, “2-theta” and “°2θ” are used interchangeably.

### DETAILED DESCRIPTION OF THE INVENTION

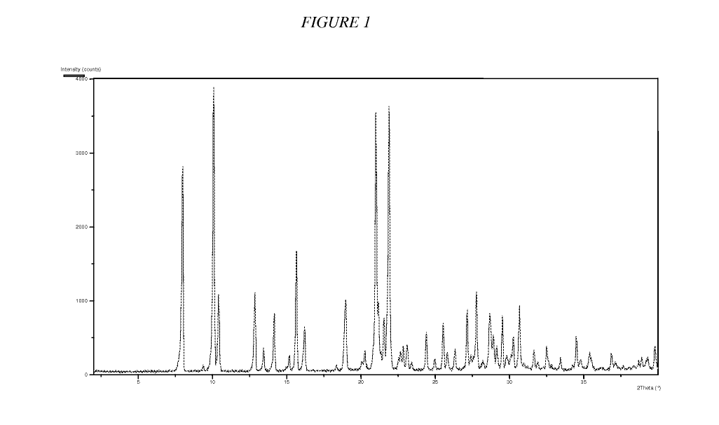

The present invention provides in one embodiment a crystalline polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine, referred to herein as Form 2. Form 2 is the only solvated crystalline form among the five forms that we have determined. Form 2 can be described by one or more solid state analytical methods, for example, by its powder X-ray diffraction pattern which is provided in FIG. 1. Powder X-ray diffraction 2-theta values for Form 2 are provided in Tables 1-3 below.

TABLE 1
2-theta Peak Values of wet Form 2 polymorph
of 7-(tert-Butyl-d9)-3-(2,5-difluorophenyl)-
6-((1-methyl-1H-1,2,4-triazol-5-yl)
methoxy)-[1,2,4]triazolo[4,3-b]pyridazine
Pos. [° 2Th.]
7.95
10.09
10.41
12.82
14.09
15.64
18.72
19.03
20.91
21.13
21.90
25.55
27.14
27.73
29.53
30.58
32.47

TABLE 2
2-theta Peak Values of air-dried Form 2 polymorph
of 7-(tert-Butyl-d9)-3-(2,5-difluorophenyl)-
6-((1-methyl-1H-1,2,4-triazol-5-yl)
methoxy)-[1,2,4]triazolo[4,3-b]pyridazine
Pos. [° 2Th.]
8.01
10.07
10.42
12.86
14.16
15.66
16.22
18.98
21.00
21.18
21.55
21.89
22.85
23.10
24.40
25.54
27.14
27.77
28.66
28.91
29.14
29.52
30.26
30.67
32.49
34.51

TABLE 3
2-theta Peak Values of vacuum-dried Form 2 polymorph
of 7-(tert-Butyl-d9)-3-(2,5-difluorophenyl)-
6-((1-methyl-1H-1,2,4-triazol-5-yl)
methoxy)-[1,2,4]triazolo[4,3-b]pyridazine.
Pos. [° 2Th.]

7.9711
9.8814
10.2988
12.9732
13.397
15.6284
16.0584
18.6984
20.7624
21.3017
21.7484
25.4395
27.077
27.927
29.4294
32.5365

In some embodiments, Form 2 is characterized as having a powder X-ray diffraction pattern having two or more peaks, in terms of 2-theta, selected from about 7.95, 10.09, 10.41, 12.82, 14.09, 15.64, 18.72, 19.03, 20.91, 21.13, 21.90, 25.55, 27.14, 27.73, 29.53, 30.58, and 32.47 degrees, at ambient temperature. In one aspect of this embodiment, Form 2 is characterized by the peaks at 2-theta values of about 7.95, 10.09, 10.41 and 20.91 degrees. In one aspect of this embodiment, Form 2 is characterized as having a powder X-ray diffraction pattern peaks, in terms of 2-theta, at each of about 7.95, 10.09, 10.41, 20.91, 12.82, 14.09, 15.64, 18.72, 19.03, 27.14, 27.73, 29.53, and 30.58 degrees, at ambient temperature. In yet further aspects, Form 2 is characterized by a powder X-ray diffraction pattern substantially as shown in FIG. 1, at ambient temperature. The relative intensities of the peaks can vary, depending upon the sample preparation technique, the sample mounting procedure, the particular instrument employed, and the morphology of the sample. Moreover, instrument variation and other factors can affect the 2-theta values. Therefore, the XRPD peak assignments for Form 2 and all other crystalline forms disclosed herein, can vary by ±0.2°.

One or more of the peaks in Tables 1-3 may be used to characterize Form 2. For example, the peak at about 7.95° 2θ of wet or “fresh” Form 2 may be used to characterize Form 2. The peak at about 10.09° 2θ may also be used to characterize Form 2 as may the peak at about 10.41° 2θ be used to characterize Form 2. Further, or more or two or more peaks selected from about 7.95° 2θ, about 10.09° 2θ, and about 10.41° 2θ may be used to characterize Form 2. The collection of peaks in Table 1 (or Table 2, or Table 3) may be used to characterize Form 2. A diffraction pattern looking substantially the same as that of FIG. 1 may also be used to characterize Form 2.

In another embodiment, Form 2 is identified by its thermal characteristics. It exhibits a weight loss of approximately 2.9% of water (when fully hydrated) as measured by TGA up to about 160° C.

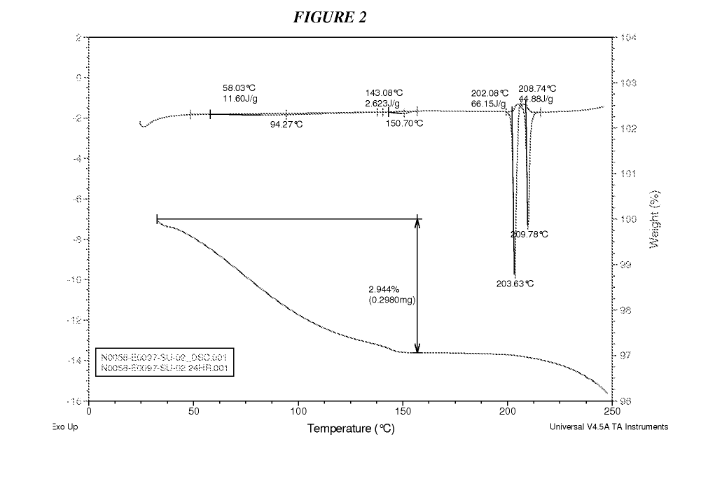

In another embodiment, Form 2 is identified by a dehydration event at about 58° C. and/or by an endotherm at about 143° C. (where Form 2 converts to Form 3). In a related aspect, Form 2 is identified by the differential calorimetric scanning (DSC) thermogram as shown in FIG. 2. For DSC, it is known that the temperatures observed will depend upon the rate of temperature change as well as sample preparation technique and the particular instrument employed. Thus, the values reported herein for Form 2 relating to melting point and DSC thermograms can vary by ±1° C.

Variation in the position of Raman peaks exists and may be due to sample conditions as well as data collection and processing. The typical variability in Raman spectra reported herein is on the order plus or minus 2.0 cm⁻¹. Thus, the use of the word “about” when referencing Raman peaks is meant to include this variability and all Raman peaks disclosed herein are intended to be reported with such variability.

In the Raman spectrum of Form 2, one or more of the peaks characterize Form 2 of the Compound of Formula I. In one embodiment, one or more of the peaks at about 1490.7 cm⁻¹, 1538.8 cm⁻¹, and 1628.2 cm⁻¹ may be used to characterize Form 2. In a further embodiment, any one or more of these Raman peaks together with any one or more of the XRPD peaks used to characterize Form 2 may be used to characterize Form 2. For example, the peaks at one or more of about 7.95° 2θ, about 10.09° 2θ, and about 10.41° 2θ together with one or more of the Raman peaks at about 1490.7 cm⁻¹, about 1538.8 cm⁻¹, and about 1628.2 cm⁻¹ may be used to characterize Form 2.

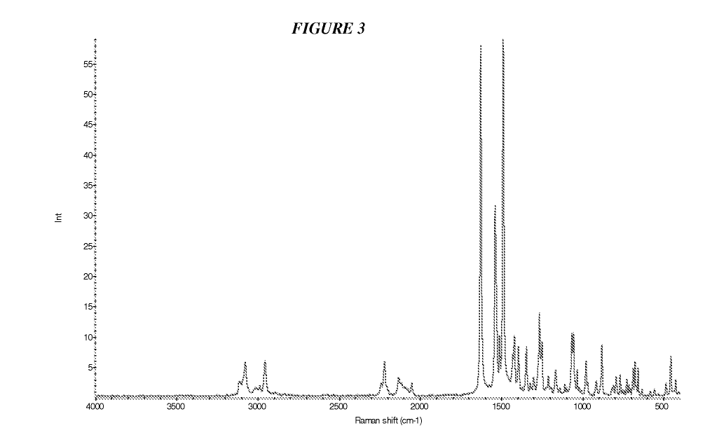

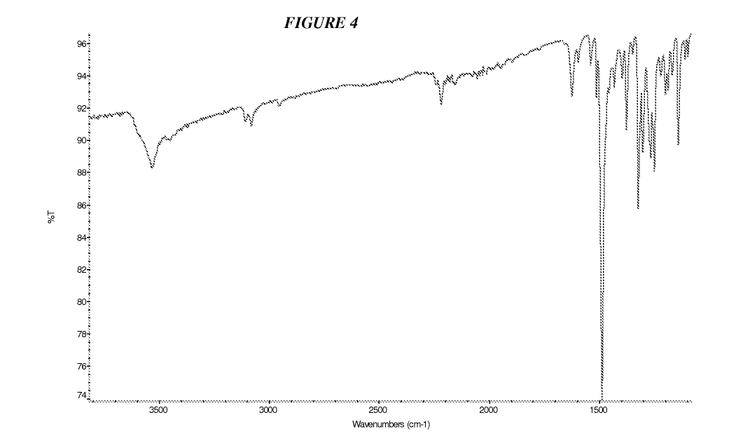

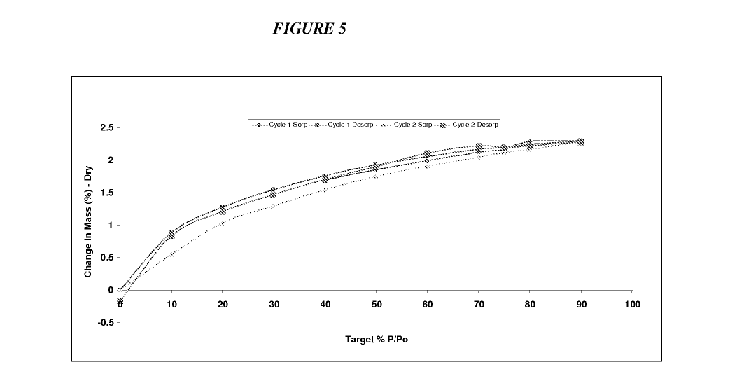

In another embodiment, Form 2 is further identified by the FT-Raman spectrum shown in FIG. 3. The pattern shows IR shift peaks at 659.2, 677.8, 689.8, 882.3, 980.1, 1035.2, 1056.4, 1067.2, 1166.5, 1250.0, 1266.5, 1347.5, 1395.8, 1421.0, 1490.7, 1538.8, 1628.2, 2219.8, 2957.1, and 3078.0 cm⁻¹, In another embodiment, Form 2 is further identified by the FT-IR spectrum shown in FIG. 4. The pattern shows IR shift peaks at 635.1, 669.4, 688.3, 709.3, 728.0, 768.4, 790.7, 826.3, 885.5, 917.3, 980.6, 1008.7, 1022.6, 1035.0, 1063.7, 1097.3, 1110.2, 1140.6, 1169.3, 1188.5, 1199.5, 1220.5, 1250.6, 1266.9, 1302.5, 1323.1, 1376.4, 1396.2, 1432.1, 1487.8, 1512.4, 1539.3, 1624.2, 2219.5, 3083.6, and 3533.9 cm⁻¹. In still another embodiment, Form 2 is further identified by the DVS isotherm plot shown in FIG. 5.

Form 2 is a hydrated crystal. DVS analysis revealed that Form 2 gained up to 2.5% wt between 0-90% relative humidity. The DVS profile along with the TGA data suggests that the hydrate is non-stoichiometric. Form 2 is physically stable at ambient conditions for at least two weeks; and at 75-97% relative humidity for five days.

Form 2 may be made by suspending Form 1, prepared as disclosed below in the Examples section, in an aqueous solvent with stirring at room temperature for a sufficient period of time, e.g., three hours and then isolating the remaining solid material. Such aqueous solvents include water, methanol, and aqueous ethanol.

Form 2 may be made by suspending Form 1 in an aqueous solvent with stirring while cycling the temperature between 5 and 40° C. for at least 48 hours. Such aqueous solvents include, but are not limited to, water, methanol, 4-methyl-2-pentanone, chlorobenzene, 1,4-dioxane, isopropyl ether, toluene, cyclohexane, t-butyl methyl ether, isopropyl acetate, cyclohexanone, 2-methoxyethyl ether, and EtOH: 5 vol % in water.

Form 2 may be produced by lyophilization of a frozen solution of Form 1 dissolved in, e.g., 50:50 dimethyl carbonate (DMC):1,4-Dioxane, 50:50 acetonitrile (ACN):1,4-Dioxane, 1:1:1 ACN:DMC:1,4-Dioxane, or 60:40 1,4-Dioxane:t-butyl alcohol (t-BuOH).

Form 2 may be prepared by evaporation at ˜30° C. of a saturated solution of Form 1 dissolved in, e.g., tetrahydrofuran, or 9% aqueous t-BuOH.

Form 2 may be prepared by rapid cooling to 5° C. of a saturated and filtered solution of Form 1 dissolved at 50° C. in, e.g., Acetone: 20 vol % in water, ethanol: 50 vol % in water, 80:20 t-BuOH:water, 70:20:10 t-BuOH:Water:trifluoroethanol, or 80:20 1,4-Dioxane:Water.

Form 2 may be prepared by cooling at a rate of 1° C./min from 25° C. to 5° C. of a saturated and filtered solution of Form 1 dissolved at 25° C. in, e.g., dichloromethand (DCM) or chlorobenzene.

Form 2 may be formed by evaporation over 2-28 days at room temperature of a saturated solution of Form 1 dissolved in, e.g., nitromethane, methyl acetate, 1,4-dioxane, DMC, EtOH: 5 vol % in water, EtOH: 5 vol % in MeOH, or toluene.

In one embodiment, the Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine has at least 98% deuterium incorporation at each position designated as deuterium in Formula I as determined by ¹H-NMR.

The invention is also directed to processes for the preparation of the Form 2 polymorph.

### Compositions

The invention also provides pyrogen-free pharmaceutical compositions comprising an effective amount of the Form 2 polymorph of this invention; and a pharmaceutically acceptable carrier. The carrier(s) are “pharmaceutically acceptable” in the sense of being not deleterious to the recipient thereof in an amount used in the medicament.

In certain embodiments, the ratio of Form 2 to other forms, including other crystalline forms such as the other crystalline forms disclosed herein, and/or amorphous forms (e.g., the ratio of the amount of Form 2 to the sum of the amounts of all other polymorphic forms of L-838417) in such pharmaceutical compositions is greater than 50:50, equal to or greater than 80:20, equal to or greater than 90:10, equal to or greater than 95:5, equal to or greater than 99:1; or 100:0.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

### Methods of Treatment

According to another embodiment, the invention provides a method of treating a treating a mammal having a disorder of the central nervous system comprising the step of administering to said mammal an effective amount of the Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine or a pharmaceutical composition comprising Form 2 polymorph of 7-(tert-Butyl-d9)-3-(2,5-difluorophenyl)-6-((1-methyl-1H-1,2,4-triazol-5-yl)methoxy)-[1,2,4]triazolo[4,3-b]pyridazine and a pharmaceutically acceptable carrier.

In one particular embodiment, the method of this invention is used to treat a disease or condition in a human patient in need thereof selected from anxiety, convulsions, neuropathic pain, inflammatory pain, and migraine-associated pain.

As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy. Effective amounts of the Form 2 polymorph of 7-(tert-Butyl-d9)-3-(2,5-difluorophenyl)-6-((1-methyl-1H-1,2,4-triazol-5-yl)methoxy)[1,2,4]triazolo[4,3-b]pyridazine can be determined by one of ordinary skill in the art. Effective doses will vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the scientific literature for L-838417. In one embodiment, an effective amount of the Form 2 polymorph can range from about 0.01 to about 5000 mg per treatment. In more specific embodiments, the range is from about 0.1 to 2500 mg, or from 0.2 to 1000 mg, or most specifically from about 1 to 500 mg. Treatment typically is administered one to three times daily.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The second therapeutic agent may be selected from any compound or therapeutic agent known to have or that demonstrates advantageous properties when administered with a compound having the same mechanism of action as L-838417.

Preferably, the second therapeutic agent is an agent useful in the treatment or prevention of a disease or condition selected from disorders of the central nervous system, including anxiety and convulsions; and neuropathic, inflammatory and migraine associated pain.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized Other potential advantages (including without limitation improved dosing regimens and/or reduced drug cost) will be apparent to those of skill in the art.

### DEFINITIONS FOR SOLVENTS

The following definitions are for solvents that are suitable in the preparation of the forms disclosed herein:

### ACN Acetonitrile

### DCM Dichloromethane

### DMC Dimethyl Carbonate

### DMSO Dimethylsulfoxide

### EtOAc Ethyl Acetate

### EtOH Ethanol

### IPA 2-Propanol

MeOAc Methyl acetate

### MeOH Methanol

n-PrOH 1-Propanol
TBME t-Butyl Methyl Ether
t-BuOH tert-Butanol

### TEE Trifluoroethanol

### THF Tetrahydrofuran

### Example 1

### Formation of 7-(tert-Butyl-d9)-3-(2,5-difluorophenyl)-6-((1-methyl-1H-1,2,4-triazol-5-yl)methoxy)-[1,2,4]triazolo[4,3-b]pyridazine Forms 1, and 2

Starting Material:

Solid 7-(tert-Butyl-d9)-3-(2,5-difluorophenyl)-6-((1-methyl-1H-1,2,4-triazol-5-yl)methoxy)[1,2,4]triazolo[4,3-b]pyridazine is prepared as depicted in Scheme 1:

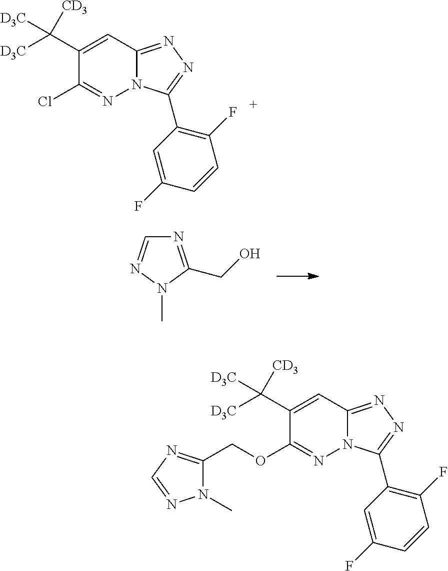

A mixture of 7-(tert-Butyl-d₉)-6-chloro-3-(2,5-difluorophenyl)-[1,2,4]triazolo[4,3-b]pyridazine (130 g, 391.8 mmol, 1 equiv) and 1-methyl-1H-1,2,4-triazol-5-yl)methanol (53 g, 470.1 mmol, 1.2 equiv) in anhydrous THF (1560 mL, 12 vol) was stirred at 20° C. under nitrogen for 5 min To this was added 1M potassium tertbutoxide in THF (470 mL, 470.1 mmol, 1.2 equiv) drop wise over 45 min while maintaining the temperature at 20-25° C. The reaction mixture was stirred for another 45 min and then diluted with water (1300 mL, 10 vol, pH 12-13) and the pH was adjusted to 7-8 with 1 HCl (30 mL). The organic solvent was removed and the aqueous layer was extracted with DCM (3×600 mL). The combined DCM layers were washed with water (1×40 mL) and brine (1×40 mL). The organic layer was concentrated to 3 volumes, solvent swapped into 5 volumes of heptanes, aged at 22° C. for 1 h. The white solid was collected by filtration to afford crude 7-(tert-Butyl-d9)-3-(2,5-difluorophenyl)-6-((1-methyl-1H-1,2,4-triazol-5-yl)methoxy)-[1,2,4]triazolo[4,3-b]pyridazine (150 g; 92.8 A %).

The crude product (150 g) was dissolved in denatured anhydrous ethanol (1950 mL, 15 vol) at 70° C. The solution was cooled to 60° C. and 7-(tert-Butyl-d9)-3-(2,5-difluorophenyl)-6-((1-methyl-1H-1,2,4-triazol-5-yl)methoxy)-[1,2,4]triazolo[4,3-b]pyridazine Form 1 seed (1.3 g) were added. The mixture was cooled to 22° C. and stirred for 5 h. The white solid was collected by filtration and dried at 45° C. under vacuum for 10 h to produce solid 7-(tert-Butyl-d9)-3-(2,5-difluorophenyl)-6-((1-methyl-1H-1,2,4-triazol-5-yl)methoxy)-[1,2,4]triazolo[4,3-b]pyridazine (“Starting Material”). Yield: 126.4 g (79%).

The synthesis of 7-(tert-Butyl-d₉)-6-chloro-3-(2,5-difluorophenyl)-[1,2,4]triazolo[4,3-b]pyridazine is described in United States patent publication No. 2010/0056529.

Form 1:

7-(tert-Butyl-d9)-3-(2,5-difluorophenyl)-6-((1-methyl-1H-1,2,4-triazol-5-yl)methoxy)[1,2,4]triazolo[4,3-b]pyridazine Form 1 was produced by dissolving solid Starting Material in anhydrous ethanol and heating to 50° C. to dissolve. The solution is then allowed to cool and resulting solids are isolated and air-dried.

Form 2:

Form 2 was prepared from Form 1 as follows. Form 1 (500.0 mg) was manually weighed into an 8-mL vial and combined with water (5.0 mL). A stir bar was added and the suspension was stirred at room temperature for 72 hrs. The white solid was isolated on a Büchner funnel by vacuum filtration and air-dried for 3 hrs.

### Example 2

### Formation of 7-(tert-Butyl-d9)-3-(2,5-difluorophenyl)-6-((1-methyl-1H-1,2,4-triazol-5-yl)methoxy)-[1,2,4]triazolo[4,3-b]pyridazine Form 2 from Form 1 with Methanol

Form 1 (100.0 mg) was manually weighed into an 8-mL vial containing a stir-bar. MeOH (2.5 mL; freshly opened bottle) was added and the suspension was stirred at room temperature for 72 hrs. The white solid was isolated on a Büchner funnel by vacuum filtration and air-dried for 3 hrs.

LM and PXRD analyses of the Form 2 samples confirmed that the material is a highly crystalline white powder. As confirmed by thermal analyses, Form 2 is a non-stoichiometric hydrate (˜2.9% wt. water loss up to 160° C. vs. theoretical wt % of 1 eq. water=4.2%). IR analysis of the volatile component released upon heating confirmed water. The DSC trace shows a broad and poorly pronounced dehydration endotherm (between RT-140° C.) followed by an endotherm/recrystallization event (at 143.1° C.), melt/recrystallization event (at 202.1° C.), and melting endotherm (at 208.7° C.). Variable-temperature PXRD (VT-PXRD) of Form 2 confirmed that the thermal transitions correspond to conversions to other crystal forms. Specifically, following dehydration, Form 2 converts to Form 3. Form 3 melts at 202.1° C. and recrystallizes to Form 4, which melts at ˜208° C. VT-PXRD indicated additional unique PXRD pattern existing at ˜210° C., however the solid isolated at that temperature was partially melted and significantly discolored/degraded.

DVS analysis revealed that Form 2 gains up to 2.5% wt between 0-90% RH. The DVS profile along with the TGA data suggests that the hydrate is non-stoichiometric.

Form 2 was observed to exhibit slight differences (peak shifts) in the PXRD patterns when wet, air-dried, or vacuum-dried (Tables 4-6). These differences were attributed to varying hydration levels of Form 2, and were supported by TGA analysis, which showed that Form 2 exposed to drying at 30° C. under vacuum for 72 hrs exhibited 1% water loss (as opposed to 2.9% for a fully hydrated Form 2). The PXRD peaks-shifts are minimized when both the wet and vacuum-dried cakes are equilibrated at ambient conditions for 3 hrs.

TABLE 4
2-theta Peak Values and intensities of wet Form 2 polymorph of
7-(tert-Butyl-d9)-3-(2,5-difluorophenyl)-6-((1-methyl-1H-1,2,4-
triazol-5-yl)methoxy)-[1,2,4]triazolo[4,3-b]pyridazine.

| Pos. [° 2Th.] | Height [cts] |
|---|---|
| 7.95 | 6837.69 |
| 10.09 | 2406.89 |
| 10.41 | 1163.86 |
| 12.82 | 183.04 |
| 14.09 | 169.64 |
| 15.64 | 741.80 |
| 18.72 | 182.20 |
| 19.03 | 208.25 |
| 20.91 | 929.85 |
| 21.13 | 185.61 |
| 21.90 | 992.75 |
| 25.55 | 224.77 |
| 27.14 | 299.09 |
| 27.73 | 161.41 |
| 29.53 | 240.19 |
| 30.58 | 146.72 |
| 32.47 | 1167.27 |

TABLE 5
2-theta Peak Values and intensities of air-dried Form 2 polymorph of
7-(tert-Butyl-d9)-3-(2,5-difluorophenyl)-6-((1-methyl-1H-1,2,4-
triazol-5-yl)methoxy)-[1,2,4]triazolo[4,3-b]pyridazine.

| Pos. [° 2Th.] | Height [cts] |
|---|---|
| 8.01 | 2499.48 |
| 10.07 | 3808.74 |
| 10.42 | 989.26 |
| 12.86 | 1049.91 |
| 14.16 | 742.54 |
| 15.66 | 1613.72 |
| 16.22 | 531.83 |
| 18.98 | 889.82 |
| 21.00 | 3498.35 |
| 21.18 | 880.09 |
| 21.55 | 718.42 |
| 21.89 | 3507.49 |
| 22.85 | 322.18 |
| 23.10 | 347.67 |
| 24.40 | 522.86 |
| 25.54 | 631.93 |
| 27.14 | 824.57 |
| 27.77 | 1036.37 |
| 28.66 | 768.41 |
| 28.91 | 471.23 |
| 29.14 | 341.36 |
| 29.52 | 741.01 |
| 30.26 | 461.84 |
| 30.67 | 881.57 |
| 32.49 | 332.59 |
| 34.51 | 436.59 |

TABLE 6
2-theta Peak Values and intensities of vacuum-dried Form 2 polymorph
of 7-(tert-Butyl-d9)-3-(2,5-difluorophenyl)-64(1-methyl-1H-1,2,4-
triazol-5-yl)methoxy)-[1,2,4]triazolo[4,3-b]pyridazine.

| Pos. [° 2Th.] | Height [cts] |
|---|---|
| 7.9711 | 6837.69 |
| 9.8814 | 2406.89 |
| 10.2988 | 1163.86 |
| 12.9732 | 183.04 |
| 13.397 | 169.64 |
| 15.6284 | 741.80 |
| 16.0584 | 182.20 |
| 18.6984 | 208.25 |
| 20.7624 | 929.85 |
| 21.3017 | 185.61 |
| 21.7484 | 992.75 |
| 25.4395 | 224.77 |
| 27.077 | 299.09 |
| 27.927 | 161.41 |
| 29.4294 | 240.19 |
| 32.5365 | 146.72 |

Solvent-wet Form 2 obtained from MeOH exhibited a more pronounced change in the PXRD pattern of the wet cake as compared to Form 2 obtained from water. In addition, several other Forms 2 products, freshly isolated from other solvents, e.g., isopropyl alcohol, exhibited slight peak shifts in their respective FT-Raman spectra. The peak shifts rapidly diminished upon air-drying at ambient for several minutes. The existence of these peak-shifts suggests the presence of a different and very unstable phase/form (e.g., another solvated or hydrated crystal form) that is only stable when in contact with mother liquor).

Form 2 was observed to precipitate from solution as large, nearly cubic or rectangular thick plates. Form 2 is physically stable when exposed to ambient conditions for at least 2 weeks; or elevated humidity (75% or 95% RH) for 5 days.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

We claim:

1. A polymorph of an optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine characterized by at least one of:
a. a powder X-ray diffraction pattern having two or more peaks expressed in degrees 2-theta±0.2° and selected from about 7.95, 10.09, 10.41, 12.82, 14.09, 15.64, 18.72, 19.03, 20.91, 21.13, 21.90, 25.55, 27.14, 27.73, 29.53, 30.58, and 32.47 degrees, at ambient temperature; or
b. a DSC thermogram showing an endotherm at about 143° C.

2. The polymorph of claim 1, characterized by a powder X-ray diffraction having peaks expressed in degrees 2-theta±0.2° at each of about 7.95, 10.09, 10.41 and 20.91 degrees.

3. The polymorph of claim 2, characterized by a powder X-ray diffraction having peaks expressed in degrees 2-theta±0.2° at each of about 7.95, 10.09, 10.41, 20.91, 12.82, 14.09, 15.64, 18.72, 19.03, 27.14, 27.73, 29.53, and 30.58 degrees.

4. The polymorph of any one of claims 1-3, wherein the optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine is 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

5. The polymorph of claim 4 having at least 98% deuterium incorporation at t-butyl position, as determined by 1H-NMR.

6. The polymorph of any one of claims 1-5 wherein the polymorph is substantially free of other forms of optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine.

7. A pharmaceutical composition comprising an effective amount of Form 2 polymorph of an optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine; and a pharmaceutically acceptable carrier.

8. The composition of claim 7, wherein the optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine is 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

9. The composition of claim 8, wherein the 7-(tert-Butyl-d9)-3-(2,5-difluorophenyl)-6-((1-methyl-1H-1,2,4-triazol-5-yl)methoxy)-[1,2,4]triazolo[4,3-b]pyridazine has at least 98% deuterium incorporation at t-butyl position, as determined by 1H-NMR.

10. The composition of claim 9, wherein the ratio of the amount of Form 2 to the sum of the amounts of other forms is equal to or greater than 80:20.

11. The composition of claim 10, wherein the ratio of the amount of Form 2 to the sum of the amounts of Form 1, Form 3, Form 4 and Form 5 is equal to or greater than 90:10.

12. A method of treating diabetic nephropathy in a patient comprising the step of administering to the patient a polymorph of claim 1.

13. A process for the preparation of the polymorph of claim 1, comprising suspending 7-(tert-Butyl-d9)-3-(2,5-difluorophenyl)-6-((1-methyl-1H-1,2,4-triazol-5-yl)methoxy)-[1,2,4]triazolo[4,3-b]pyridazine in an aqueous solvent with stirring at room temperature and isolating any solid material.

14. The process of claim 13, wherein the aqueous solvent is selected from water, aqueous ethanol, aqueous methanol and aqueous isopropanol.

15. The polymorph of claim 1, wherein the polymorph is substantially free of amorphous 7-(tert-Butyl-d9)-3-(2,5-difluorophenyl)-6-((1-methyl-1H-1,2,4-triazol-5-yl)methoxy)-[1,2,4]triazolo[4,3-b]pyridazine.

16. Form 2 of optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine with a water content of about 2.9%.

17. Form 2 of optionally deuterated 7-(tert-Butyl)-3-(2,5-difluorophenyl)-6-((1-methyl-1H-1,2,4-triazol-5-yl)methoxy)-[1,2,4]triazolo[4,3-b]pyridazine with a water content of less than 2.9%.

18. Form 2 of claim 16 or 17 having powder X-ray diffraction peaks at one or more of about 7.95, 10.09, 10.41° 2θ±0.2° 2θ.

19. Form 2 of one of claim 16, 17 or 18 having Raman peaks at one or more of about 1490.7 cm⁻¹, 1538.8 cm⁻¹, and 1628.2 cm⁻¹.

20. Form 2 of claim 19 having Raman peaks at one or more of about 1490.7 cm⁻¹, 1538.8 cm⁻¹, and 1628.2 cm⁻¹.

21. Form 2 of any one of claims 16-20 having at least 98% deuterium incorporation at t-butyl position, as determined by 1H-NMR.

22. Form 2 of any one of claims 16-21 wherein Form 2 is substantially free of other polymorphic forms of L-838417.

23. Form 2 of any one of claims 16-22 wherein Form 2 is substantially free of Form 1, Form 3, Form 4 and Form 5 of L-838417.

24. A pharmaceutical composition comprising an effective amount of Form 2 of any one of claims 16 to 23.

25. The composition of claim 24, wherein the ratio of the amount of Form 2 to the sum of the amounts of all other polymorphic forms of L-838417 is equal to or greater than 80:20.

26. The composition of claim 24, wherein the ratio of the amount of Form 2 to the sum of the amounts of Form 1, Form 3, Form 4 and Form 5 is equal to or greater than 80:20.

27. The composition of claim 26, wherein the ratio of the amount of Form 2 to the sum of the amounts of Form 1, Form 3, Form 4 and Form 5 is equal to or greater than 90:10.
